(19) United States
(12) Patent Application Publication
(10) Pub. No.: US 2025/0339589 A1
(43) Pub. Date: Nov. 6, 2025

(54) MEDICAL DEVICES

(71) Applicant: Unomedical A/S, Lejre (DK)

(72) Inventors: Jeffrey Cafmeyer, Columbus, OH (US); Erik Edwards, Columbus, OH (US); Neal Carty, Las Vegas, NV (US); Lisa Jaunet van Kooten, Lejre (DK)

(21) Appl. No.: 19/072,932

(22) Filed: Mar. 6, 2025

Related U.S. Application Data
(63) Continuation of application No. PCT/EP2023/073832, filed on Aug. 30, 2023.
(60) Provisional application No. 63/404,298, filed on Sep. 7, 2022.

(30) Foreign Application Priority Data
Oct. 20, 2022 (GB) 2215512.1

(51) Int. Cl. A61L 29/08; A61M 5/158; A61M 25/00

(52) U.S. Cl. CPC A61L 29/085; A61M 5/158; A61M 25/0045; A61L 2420/02; A61M 2207/00

(57) ABSTRACT

The invention provides a medical device comprising a fluoropolymer surface comprising at least one hyaluronic acid species.

### TECHNICAL FIELD OF THE INVENTION

The present invention relates to medical devices comprising fluoropolymer surfaces containing hyaluronic acid species. The present invention also relates to the use of a hyaluronic acid species as a protein-repellent in/on a medical device.

### BACKGROUND TO THE INVENTION

Cannulas and catheters are indispensable in the medical field and are inserted into the body, often for the delivery or removal of fluid. The material and configuration of such medical devices vary enormously depending on their intended use. Typical uses of cannulas and catheters include cardiovascular, urological, gastrointestinal, neurovascular, and ophthalmic applications.

There has been recent interest in constructing such medical devices using fluoropolymer materials, in particular polytetrafluoroethylene (PTFE). Such fluoropolymers are advantageous for use in medical applications due to their favourable mechanical properties and excellent chemical stability under biological conditions.

However, when such insertable medical devices are introduced into the body, foreign body responses can occur—i.e. where a patient's body identifies the medical device as foreign and rejects it. Such responses can begin as early as on insertion of the medical device into the body, which can cause inflammation and trigger an immediate rush of inflammatory-mediating cells and proteins to the area of insertion. Proteins typically then non-specifically adsorb to the medical device surface, forming a protein layer which becomes a provisional matrix, through which cells and bacteria gathering in the area can identify and interact with the foreign body.

Foreign body responses can ultimately cause numerous problems, including device clogging and infection. The negative impacts are often exacerbated when such medical devices are inserted by untrained personnel—e.g. by a user themselves in the absence of a medical professional. Furthermore, acute responses are particularly common for medical devices which are inserted subcutaneously or intravenously into the body, and these can have many harmful and even life-threatening consequences.

Medical device surface coatings and additives have been investigated to overcome the above issues. However, these have not been without disadvantages. Further, very few, if any, of these coatings and additives have been successful with fluoropolymer medical devices, especially PTFE medical devices. The chemical inertness of fluoropolymers makes them notoriously difficult to chemically modify or coat with an additive. Such polymers also display practical incompatibility with a vast range of chemistry commonly employed in medical device surface coatings and additives.

There exists a need for medical devices with further safeguards to ameliorate one or more of the above issues; in particular safeguards which are suitable for use with fluoropolymer-containing medical devices.

It is an aim of embodiments of the present invention to address or ameliorate one or more of the above problems of the prior art. In particular, it is an aim of embodiments of the present invention to provide a fluoropolymer-containing medical device which has one or more of the following advantages:
Excellent mechanical properties.
A non-stick and/or lubricious surface. Easy to insert and remove from the body.
Antibiofouling properties.
Protein-repellent properties/provides minimal protein adsorption.
Low risk of initiating foreign body responses or delayed foreign body response initiation.
Safe to use. No chemical leaching from the medical device, especially when inserted into the body.
Safe and effective even when implanted into the body for long periods of time.
Ease of manufacture.

It is also an aim of embodiments of the present invention to overcome or mitigate at least one problem of the prior art, whether expressly described herein or not.

### SUMMARY OF THE INVENTION

According to a first aspect of the invention, there is provided a medical device comprising a fluoropolymer surface comprising at least one hyaluronic acid species.

At least one hyaluronic acid species on a fluoropolymer surface provides the surface of the medical device with a high level of resistance to protein adsorption and adhesion. The hyaluronic acid species protects the fluoropolymer surface from being targeted by foreign body responses. The hyaluronic acid species also displays high chemical compatibility with the fluoropolymer medical device surface; the fluoropolymer surface displays excellent mechanical properties and provides for a lubricious non-stick surface.

In some embodiments, the hyaluronic acid species is present as a coating on the fluoropolymer surface. In some embodiments, at least 75% of the coating is the hyaluronic acid species, or at least 76, 77, 78, 79, 80, 81, 82, 83, 84, 85, 86, 87, 88, 89, 90, 91, 92, 93, 94, 95, 96, 97, 98, 99, or 100% of the coating is the hyaluronic acid species. In some embodiments, no greater than 95, 90, 85, or no greater than 80% of the coating is the hyaluronic acid species.

In some embodiments, the fluoropolymer is independently chosen from: polytetrafluoroethylene (PTFE), polyvinylfluoride, polyvinylidene fluoride, polychlorotrifluoroethylene, a perfluoroalkoxy polymer, fluorinated ethylene-propylene, polyethylenetetrafluoroethylene, polyethylenechlorotrifluoroethylene, a perfluoroelastomer, a fluoroelastomer, perfluoropolyether, perfluorosulfonic acid, perfluoropolyoxetane, and combinations, blends or copolymers thereof.

In some embodiments, the fluoropolymer is independently selected from the group consisting of: polytetrafluoroethylene (PTFE), polyvinylfluoride, polyvinylidene fluoride, polychlorotrifluoroethylene, a perfluoroalkoxy polymer, fluorinated ethylene-propylene, polyethylenetetrafluoroethylene, polyethylenechlorotrifluoroethylene, a perfluoroelastomer, a fluoroelastomer, perfluoropolyether, perfluorosulfonic acid, perfluoropolyoxetane, and combinations, blends or copolymers thereof.

The fluoropolymer may be independently chosen from: PTFE, fluorinated ethylene-propylene, polyvinylidene fluoride, and combinations, blends or copolymers thereof.

The fluoropolymer may be independently selected from the group consisting of: PTFE, fluorinated ethylene-propylene, polyvinylidene fluoride, and combinations, blends or copolymers thereof.

In a particularly preferred embodiment, the fluoropolymer is or comprises PTFE. PTFE provides excellent mechanical properties and demonstrates good compatibility with the hyaluronic acid species, despite its high fluorine-to-carbon ratio.

At least one hyaluronic acid species may be independently chosen from: hyaluronan, heparin, heparan, chondroitin, keratan, dermatan, and derivatives and/or combinations thereof. At least one hyaluronic acid species may be a hyaluronic acid sulfate derivative, preferably of a species listed above. In preferred embodiments, at least one hyaluronic acid species comprises hyaluronan or a derivative thereof.

At least one hyaluronic acid species may be independently selected from the group consisting of: hyaluronan, heparin, heparan, chondroitin, keratan, dermatan, and derivatives and/or combinations thereof. At least one hyaluronic acid species may be a hyaluronic acid sulfate derivative, preferably of a species listed above. In preferred embodiments, at least one hyaluronic acid species comprises hyaluronan or a derivative thereof.

In some embodiments, fluoropolymer surface comprises 2 or at least 2 different hyaluronic acid species, or 3 or at least 3, 4 or at least 4, 5 or at least 5, 6 or at least 6, 7 or at least 7, 8 or at least 8, 9 or at least 9, or 10 or at least 10 different hyaluronic acid species. In some embodiments, the fluoropolymer surface comprises no greater than 10 different hyaluronic acid species, or no greater than 9, 8, 7, 6, 5, 4, 3, or no greater than 2 different hyaluronic acid species. In some embodiments, the fluoropolymer comprises a single hyaluronic acid species.

In some embodiments, at least one hyaluronic acid species is an oligomer or polymer. At least one hyaluronic acid species may be a homopolymer or a copolymer. At least one hyaluronic acid polymer may be independently chosen from: a linear polymer, a branched polymer, a graft polymer, a dendritic polymer, a star polymer, a dendronized polymer, a comb polymer, a polymer brush, a ladder polymer, and combinations thereof. At least one hyaluronic acid polymer may be independently selected from the group consisting of: a linear polymer, a branched polymer, a graft polymer, a dendritic polymer, a star polymer, a dendronized polymer, a comb polymer, a polymer brush, a ladder polymer, and combinations thereof.

At least one hyaluronic acid species may be independently selected from: an anionic species, a cationic species, a non-ionic species, and combinations thereof.

At least one hyaluronic acid species preferably comprises at least one repeating disaccharide, preferably a repeating disaccharide structure of glucoronic acid or a derivative thereof and N-acetylglucosamine or a derivative thereof. At least one hyaluronic acid species preferably contains a repeating disaccharide structure of D-glucoronic acid or a derivative thereof and N-acetyl-D-glucosamine or a derivative thereof. The glucuronic acid or derivative thereof and the N-acetylglucosamine or derivative thereof may be joined by β glycosidic bonds. The glucuronic acid or derivative thereof and the N-acetylglucosamine or derivative thereof may be joined by alternating glucoronidic and glucosaminidic bonds. The glucuronic acid or derivative thereof and the N-acetylglucosamine or derivative thereof may be joined by alternating β1-+3 and β1->4 glycosidic bonds, preferably β1->3 glucoronidic and β1->4 glucosaminidic bonds.

In some embodiments, at least one hyaluronic acid species comprises at least 1 disaccharide unit, or at least 2, 3, 4, 5, 6, 7, 8, 9, or at least 10 disaccharide units, or at least 20, 30, 40, 50, 75, 100, 150, 200, 250, 300, 350, 400, 450, 500, 750, 1000, 2000, 3000, 4000, 5000, 6000, 7000, 8000, 9000, or at least 10,000 disaccharide units. At least one hyaluronic acid species may comprise no greater than 50,000 disaccharide units, or no greater than 45,000, 40,000, 35,000, or no greater than 30,000 disaccharide units.

At least one hyaluronic acid species may have a molecular weight of at least 400 Da (Daltons), or at least 500, 600, 700, 800, 900, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or at least 10,000, 15,000, 20,000, 25,000, 30,000, 35,000, 40,000, 45,000, or at least 50,000, or at least 100,000, or at least 250,000, 500,000, 750,000, or at least 1,000,000 Da. At least one hyaluronic acid species may have a molecular weight of no greater than 20 MDa, or of no greater than 19, 18, 17, 16, 15, 14, 13, 12, 11, 10, 9, 8, 7, 6, 5, 4, 3, 2, or of no greater than 1 MDa. At least one hyaluronic acid species may have a molecular weight of between about 5,000 to about 20,000,000 Da, or of between about 10,000 to about 12,000,000 Da, or between about 1,000,000 to about 10,000,000 Da. At least one hyaluronic acid species may have a molecular weight of between about 400 to about 5,000 Da, or of between about 400 to about 4,000 Da. At least one hyaluronic acid species may have a molecular weight of between about 1,000 to about 50,000 Da, or between about 2,000 to about 45,000, or between about 3,000 to about 40,000, or between about 3,000 to about 40,000, or between about 4,000 to about 35,000, or between about 5,000 to about 30,000, or between about 5,500 to about 25,000, or between about 6,000 to about 20,000 Da. At least one hyaluronic acid derivative may have a molecular weight of between about 5,000 to about 500,000 Da, or between about 10,000 to about 400,000, or between about 15,000 to about 300,000, or between about 20,000 to about 200,000 Da.

In some embodiments, at least one hyaluronic acid species is adsorbed to the fluoropolymer surface. At least one hyaluronic acid species may be physisorbed to the fluoropolymer surface.

In preferred embodiments, at least one hyaluronic acid may be chemisorbed to the fluoropolymer surface.

In some embodiments, at least one hyaluronic acid species is covalently bonded to the fluoropolymer surface.

In some preferred embodiments, there is provided a medical device comprising a fluoropolymer surface comprising at least one hyaluronic acid species that is covalently bonded to the fluoropolymer surface.

At least one hyaluronic acid species may be ionically and/or electrostatically bonded to the fluoropolymer surface. In some embodiments, at least one hyaluronic acid species is both covalently and ionically/electrostatically bonded to the fluoropolymer surface.

In some embodiments, at least one hyaluronic acid species is directly bonded to the fluoropolymer surface. At least one hyaluronic acid species may be bonded to the fluoropolymer surface via a linker. At least one hyaluronic acid species may be bonded to the linker by a covalent bonding method. At least one hyaluronic acid species may be bonded to the linker by an ionic and/or electrostatic bonding method. The fluoropolymer surface may be bonded to the linker by a covalent bonding method. The fluoropolymer surface may be bonded to the linker by an ionic and/or electrostatic bonding method. In some embodiments, at least one hyaluronic acid species is bonded to the linker by a covalent or ionic/electrostatic bonding method and the linker is bonded to the fluoropolymer surface by the same bonding method. Alternatively, at least one hyaluronic acid species may be bonded to the linker by a covalent or ionic/electrostatic bonding method and the linker may be bonded to the fluoropolymer surface by the opposite bonding method. In some embodiments, at least one hyaluronic acid species is bonded to the linker by a covalent bonding method and the linker is bonded to the fluoropolymer surface by a covalent or ionic/electrostatic bonding method. In some embodiments, at least one hyaluronic acid species is bonded to the linker by an ionic and/or electrostatic bonding method and the linker is bonded to the fluoropolymer surface by a covalent or ionic/electrostatic bonding method.

In some preferred embodiments, there is provided a medical device comprising a fluoropolymer surface comprising at least one hyaluronic acid species that is bonded to the fluoropolymer surface via a linker, and the hyaluronic acid species is bonded to the linker by a covalent bonding method and the linker is bonded to the fluoropolymer surface by the same bonding method.

In some embodiments, at least one hyaluronic acid species is bonded to the fluoropolymer surface and/or to a linker via at least one carboxyl group on the hyaluronic acid species.

In some preferred embodiments, there is provided a medical device comprising a fluoropolymer surface comprising at least one hyaluronic acid species that is bonded to the fluoropolymer surface via at least one carboxyl group on the hyaluronic acid species.

Bonding the hyaluronic acid species via a carboxyl group does not negatively affect the structure and properties of the hyaluronic acid species—in particular the anti-inflammatory properties of the species remain unaffected. At least one hyaluronic acid may be bonded to the fluoropolymer surface and/or to a linker via an electrostatic and/or ionic bond through a carboxylate group on the hyaluronic acid species. At least one hyaluronic acid species may be bonded to the fluoropolymer surface and/or to a linker via an ester and/or amide bond formed through at least one carboxyl group on the hyaluronic acid species.

In some preferred embodiments, there is provided a medical device comprising a fluoropolymer surface comprising at least one hyaluronic acid species that is bonded to the fluoropolymer surface and/or to a linker via an ester and/or amide bond formed through at least one carboxyl group on the hyaluronic acid species.

In some preferred embodiments, there is provided a medical device comprising a fluoropolymer surface comprising at least one hyaluronic acid species that is bonded to the fluoropolymer surface via an ester and/or amide bond formed through at least one carboxyl group on the hyaluronic acid species.

In some preferred embodiments, there is provided a medical device comprising a fluoropolymer surface comprising at least one hyaluronic acid species that is bonded to the fluoropolymer surface via a linker and the hyaluronic acid species is bonded to the linker via an ester and/or amide bond formed through at least one carboxyl group on the hyaluronic acid species.

At least one hyaluronic acid species may be bonded to the fluoropolymer surface and/or to a linker via a hydroxyl group on the hyaluronic acid species. The hydroxyl group may preferably be a C6-hydroxyl group. At least one hyaluronic acid species may be bonded to the fluoropolymer surface and/or to a linker via an electrostatic and/or ionic bond through an alkoxide on the hyaluronic acid species, which may be a C6-alkoxide. In some embodiments, at least one hyaluronic acid species is bonded to the fluoropolymer surface and/or to a linker via an ester bond formed using at least one hydroxyl group on the hyaluronic acid species. In some embodiments, at least one hyaluronic acid species is bonded to the fluoropolymer surface and/or linker via an ether bond formed using at least one hydroxyl group on the hyaluronic acid species.

In preferred embodiments, the fluoropolymer surface is an activated fluoropolymer surface. Throughout this specification, the term “fluoropolymer surface” may be used to refer to an “activated fluoropolymer surface”. The activated fluoropolymer surface may comprise at least one electronegative atom. The fluoropolymer surface may be oxidised and may comprise at least one oxygen-containing moiety. In some embodiments, at least one hyaluronic acid species and/or linker is covalently bonded to the activated fluoropolymer surface via at least one oxygen-containing moiety on the fluoropolymer surface.

In some preferred embodiments, there is provided a medical device comprising a fluoropolymer surface comprising at least one hyaluronic acid species, wherein the fluoropolymer surface is an activated fluoropolymer surface and at least one hyaluronic acid species is covalently bonded to the activated fluoropolymer surface via at least one oxygen-containing moiety on the fluoropolymer surface and/or at least one hyaluronic acid species is bonded to the fluoropolymer surface via a linker and the linker is covalently bonded to the activated fluoropolymer surface via at least one oxygen-containing moiety on the fluoropolymer surface.

In some preferred embodiments, there is provided a medical device comprising a fluoropolymer surface comprising at least one hyaluronic acid species, wherein the fluoropolymer surface is an activated fluoropolymer surface and at least one hyaluronic acid species is covalently bonded to the activated fluoropolymer surface via at least one oxygen-containing moiety on the fluoropolymer surface.

In some preferred embodiments, there is provided a medical device comprising a fluoropolymer surface comprising at least one hyaluronic acid species, wherein the fluoropolymer surface is an activated fluoropolymer surface and at least one hyaluronic acid species is bonded to the fluoropolymer surface via a linker and the linker is covalently bonded to the activated fluoropolymer surface via at least one oxygen-containing moiety on the fluoropolymer surface.

At least one hyaluronic acid species and/or linker may be covalently bonded to the activated fluoropolymer surface through an ether and/or ester bond with at least one oxygen-containing moiety on the fluoropolymer surface.

At least one hyaluronic acid species and/or linker may be ionically and/or electrostatically bonded to the activated fluoropolymer surface. In some embodiments, at least one hyaluronic acid species and/or linker is bonded to the activated fluoropolymer surface via a hydrogen bonding interaction with at least one oxygen-containing moiety on the activated fluoropolymer surface.

In some preferred embodiments, there is provided a medical device comprising a fluoropolymer surface comprising at least one hyaluronic acid species, wherein the fluoropolymer surface is an activated fluoropolymer surface and at least one hyaluronic acid species is bonded to the activated fluoropolymer surface via a hydrogen bonding interaction with at least one oxygen-containing moiety on the activated fluoropolymer surface and/or at least one hyaluronic acid species is bonded to the fluoropolymer surface via a linker and the linker is bonded to the activated fluoropolymer surface via a hydrogen bonding interaction with at least one oxygen-containing moiety on the activated fluoropolymer surface.

In some preferred embodiments, there is provided a medical device comprising a fluoropolymer surface comprising at least one hyaluronic acid species, wherein the fluoropolymer surface is an activated fluoropolymer surface and at least one hyaluronic acid species is bonded to the activated fluoropolymer surface via a hydrogen bonding interaction with at least one oxygen-containing moiety on the activated fluoropolymer surface.

In some preferred embodiments, there is provided a medical device comprising a fluoropolymer surface comprising at least one hyaluronic acid species, wherein the fluoropolymer surface is an activated fluoropolymer surface and at least one hyaluronic acid species is bonded to the fluoropolymer surface via a linker and the linker is bonded to the activated fluoropolymer surface via a hydrogen bonding interaction with at least one oxygen-containing moiety on the activated fluoropolymer surface.

In such embodiments, the hyaluronic acid species and/or linker may act as a hydrogen bond donor. The activated fluoropolymer surface may act as a hydrogen bond acceptor.

In some embodiments, at least one hyaluronic acid species is bonded to a linker by a bonding type independently chosen from: an ester bond, an amide bond, an ether bond, and an ionic bond; and the linker is bonded to the fluoropolymer surface by a bonding type independently chosen from: a hydrogen bond, an ether bond, and an ionic bond.

In some embodiments, at least one hyaluronic acid species is bonded to a linker by a bonding type independently selected from the group consisting of: an ester bond, an amide bond, an ether bond, and an ionic bond; and the linker is bonded to the fluoropolymer surface by a bonding type independently selected from the group consisting of: a hydrogen bond, an ether bond, and an ionic bond.

In some embodiments, at least one hyaluronic acid species is bonded to a linker by an ionic bond, preferably through at least one carboxylate or alkoxide group of the hyaluronic acid species; and the linker is bonded to the fluoropolymer surface by a hydrogen bond, preferably through at least one oxygen-containing moiety on the fluoropolymer surface, wherein the oxygen-containing moiety preferably acts as a hydrogen bond acceptor. In some embodiments, at least one hyaluronic acid species is bonded to a linker by an amide bond, preferably through at least one carboxyl group of the hyaluronic acid species; and the linker is bonded to the fluoropolymer surface by a hydrogen bond, preferably through at least one oxygen-containing moiety on the fluoropolymer surface, wherein the oxygen-containing moiety preferably acts as a hydrogen bond acceptor. In such embodiments, the linker may be derived from a linking compound comprising at least one hydrogen bond donor and at least one group independently selected from: a nucleophilic group and an ionisable moiety. A single group may function as both a nucleophilic group and an ionisable moiety. In a particular embodiment, the linker may be derived from dopamine or a derivative thereof.

In some embodiments, at least one hyaluronic acid species is bonded to a linker by an ether bond, preferably through at least one hydroxyl group of the hyaluronic acid species; and the linker is bonded to the fluoropolymer surface by an ether bond, preferably through at least one oxygen containing-moiety on the surface. In some embodiments, at least one hyaluronic acid species is bonded to a linker by an ester bond, preferably through at least one carboxyl group or at least one hydroxyl group of the hyaluronic acid species; and the linker is bonded to the fluoropolymer surface by an ether bond, preferably through at least one oxygen containing-moiety on the surface. In such embodiments, the linker may be derived from a linking compound comprising two or at least two electrophilic groups. The electrophilic groups may be the same or different, preferably different. At least one electrophilic group may comprise a carbon atom bonded to a halogen and at least one electrophilic group may comprise a carbon atom bonded to an oxygen atom. In embodiments, the linker is derived from an epihalohydrin, preferably epichlorohydrin. In other such embodiments, the linker may be derived from a linking compound comprising a polymerizable moiety and an electrophilic moiety. The linker may be derived from a linking compound comprising a polymerizable unsaturated group, preferably an acrylate or methacrylate group. The electrophilic moiety may preferably comprise an electrophilic carbon centre. In embodiments, the linker may be derived from glycidyl acrylate and/or a glycidyl alkacrylate. In a particular embodiment, the linker is derived from glycidyl methacrylate.

In some embodiments, at least one hyaluronic acid species is bonded to a linker by an ionic bond, preferably through at least one carboxylate or alkoxide group of the hyaluronic acid species; and the linker is bonded to the fluoropolymer surface by an ionic bond, preferably through at least one negatively charged oxygen-containing moiety on the fluoropolymer surface. In some embodiments, at least one hyaluronic acid species is bonded to a linker by an amide bond, preferably through at least one carboxyl group of the hyaluronic acid species; and the linker is bonded to the fluoropolymer surface by an ionic bond, preferably through at least one negatively charged oxygen-containing moiety on the fluoropolymer surface.

In some embodiments, the linker is derived from a linking compound comprising at least one amine group. The amine group may form a covalent bond with the fluoropolymer surface and/or with at least one hyaluronic acid species. In some embodiments, at least one amine group of the linking compound forms an amide bond to at least one hyaluronic acid species and/or to the fluoropolymer surface, preferably to at least one hyaluronic acid species. In some embodiments, the linker comprises at least one cationic ammonium group, which is preferably a protonated amine group of the linking compound. The cationic ammonium group may form an ionic and/or electrostatic bond with the fluoropolymer surface and/or with at least one hyaluronic acid species. In some embodiments, the cationic ammonium group forms an ionic and/or electrostatic bond with a carboxylate group on at least one hyaluronic acid species. In some embodiments, the cationic ammonium group forms an ionic and/or electrostatic bond with an oxygen-containing moiety, preferably a negatively charged oxygen-containing moiety on the treated fluoropolymer surface.

In some embodiments, the linker is derived from a linking compound comprising a bi- or poly-functional molecule comprising at least two reactive functional groups.

In some preferred embodiments, there is provided a medical device comprising a fluoropolymer surface comprising at least one hyaluronic acid species that is bonded to the fluoropolymer surface via a linker, and the linker is derived from a linking compound comprising a bi- or poly-functional molecule comprising at least two reactive groups.

A reactive functional group may be independently selected from: a nucleophilic group and an electrophilic group. In some embodiments, the linking compound comprises at least one nucleophilic group and at least one electrophilic group. The linking compound may comprise at least two nucleophilic groups or at least two electrophilic groups. The nucleophilic group may comprise at least one electronegative element, which may be independently chosen from: oxygen, nitrogen, sulfur, and combinations thereof. The nucleophilic group may comprise at least one electronegative element, which may be independently selected from the group consisting of: oxygen, nitrogen, sulfur, and combinations thereof. In some embodiments, the electrophilic group comprises an electrophilic carbon centre, which may comprise a carbon atom bonded to an electronegative atom. The carbon atom may be bonded to an electronegative atom independently selected from: a halogen and an oxygen. The electrophilic group may comprise an epoxide group. In some embodiments, the linking compound comprises at least two groups independently selected from: an electrophilic group, a nucleophilic group, a hydrogen bond donor, an ionisable moiety, a polymerizable moiety (preferably an unsaturated group), and combinations thereof.

In preferred embodiments, the linking compound comprises at least one diamine. At least one diamine may comprise an aliphatic diamine and/or an aromatic diamine. The aromatic diamine may comprise a phenylenediamine and/or a xylylenediamine. The aliphatic diamine may comprise at least one diamine independently selected from: a linear aliphatic diamine, a branched aliphatic diamine, a cyclic aliphatic diamine, and combinations. The diamine may preferably comprise a linear diamine, preferably a linear aliphatic diamine. The diamine may preferably be an alkylenediamine, preferably a linear alkylenediamine. The alkylenediamine may comprise a C1-C15 alkylenediamine, C1-C10 alkylenediamine, preferably C1-C6 alkylenediamine, or more preferably C1-C4 alkylenediamine. In a particularly preferred embodiment, the alkylenediamine comprises ethylenediamine.

Bonding the hyaluronic acid species to the fluoropolymer surface via such diamine linking compounds results in a surface which displays an enhanced level of resistance to protein adsorption. The diamine linker chemistry is well-adapted for use with fluoropolymers, including PTFE. Further, the structure and function of the hyaluronic acid species remains unaffected.

In embodiments wherein the linking compound comprises a diamine, one amine group of the diamine may take the form of an ammonium cation and may be ionically and/or electrostatically bonded with the activated fluoropolymer surface. Said ammonium cation may be ionically and/or electrostatically bonded with an oxygen-containing moiety on the activated fluoropolymer surface. The second amine group of the diamine may take the form of an ammonium cation and may be ionically and/or electrostatically bonded with at least one hyaluronic acid species, preferably with an oxygen-containing moiety on the hyaluronic acid species. The oxygen-containing moiety on the hyaluronic acid species may comprise a carboxylate and/or an alkoxide group. Alternatively, the second amine group may form an amide bond with at least one carboxyl moiety on at least one hyaluronic acid species.

In some embodiments, the linking compound comprises a polymerizable moiety and an electrophilic moiety. The linking compound may comprise a polymerizable unsaturated group, preferably an acrylate or methacrylate group. The electrophilic moiety may preferably comprise an electrophilic carbon centre. In embodiments, the linking compound comprises glycidyl acrylate and/or a glycidyl alkacrylate. In a particular embodiment, the linking compound comprises glycidyl methacrylate.

In some embodiments, the linking compound comprises two or at least two electrophilic groups. The electrophilic groups may be the same or different, preferably different. At least one electrophilic group may comprise a carbon atom bonded to a halogen and at least one electrophilic group may comprise a carbon atom bonded to an oxygen atom. In embodiments, the linking compound comprises an epihalohydrin, preferably epichlorohydrin.

In some embodiments, the linking compound comprises at least one hydrogen bond donor and at least one group independently selected from: a nucleophilic group and an ionisable moiety. A single group may function as both a nucleophilic group and an ionisable moiety. In a particular embodiment, the linking compound comprises dopamine.

In some embodiments, the hyaluronic acid species is present at a total concentration of at least 0.1, 0.2, 0.3, 0.4, or of at least 0.5 wt. % of the medical device. The hyaluronic acid species may be present at a total concentration of no greater than 20 wt. % of the medical device, or no greater than 15, 10, 5, 4, 3, 2, 1, 0.75 or of no greater than 0.5 wt % of the medical device. The hyaluronic acid species may be present at a total concentration of between 0.1-20 wt. %, or between 0.5-15 wt. % or 0.5-5 wt. % of the medical device.

In some preferred embodiments, there is provided a medical device comprising a fluoropolymer surface comprising at least one hyaluronic acid species that is present at a total concentration of at least 0.5 wt. % of the medical device.

The hyaluronic acid species is preferably present at and/or on the fluoropolymer surface. In some embodiments, the hyaluronic acid species is present at and/or on at least 5% of the total area of the fluoropolymer surface, or at least 10, 15, 20, 25, 30, 35, 40, 45, 50, 55, 60, 65, 70, 75, 80, 85, 90, 95, 96, 97, 98, or at least 99% of the total area of the fluoropolymer surface, preferably at least 75% or at least 90% of the total area of the fluoropolymer surface or between 75% and 100% of the total area of the fluoropolymer surface. In some embodiments, the hyaluronic acid species is present at and/or on no greater than 95% of the total area of the fluoropolymer surface, or no greater than 90, 85, or no greater than 80% of the total area of the fluoropolymer surface.

In some embodiments, the hyaluronic acid species comprises a layer that is on the fluoropolymer surface. In some embodiments, the hyaluronic acid species is adsorbed to the fluoropolymer surface to form the layer. In some embodiments, the layer is a layer of coating comprising the hyaluronic acid species, which may be as described in statements of invention above.

In some embodiments, at least 75% of the layer comprising the hyaluronic acid species, or at least 76, 77, 78, 79, 80, 81, 82, 83, 84, 85, 86, 87, 88, 89, 90, 91, 92, 93, 94, 95, 96, 97, 98, 99, or 100% of the layer is the hyaluronic acid species. In some embodiments, no greater than 95, 90, 85, or no greater than 80% of the layer comprising the hyaluronic acid species is the hyaluronic acid species.

In some embodiments, the layer comprising the hyaluronic acid species has a thickness of at least 1 μm, or of at least 2, 3, 4, 5, 6, 7, 8, 9, 10, 15, 20, 25, 30, 35, 40, 45, or of at least 50 μm. The layer comprising the hyaluronic acid species may have a thickness of no more than 10000 μm, or of no more than 9000, 8000, 7000, 6000, 5000, 4000, 3000, 2000, 1000, 900, 800, 700, 600, 500, 400, or of no more than 300 μm.

In some embodiments, the hyaluronic acid species comprises an integral part of the fluoropolymer surface. The hyaluronic acid species may comprise an integral part of the fluoropolymer surface over at least 5% of the total area of the fluoropolymer surface, or at least 10, 15, 20, 25, 30, 35, 40, 45, 50, 55, 60, 65, 70, 75, 80, 85, 90, 95, 96, 97, 98, or at least 99% of the total area of the fluoropolymer surface, preferably at least 75% or at least 90% of the total area of the fluoropolymer surface or between 75% and 100% of the total area of the fluoropolymer surface. In some embodiments, the hyaluronic acid species forms an integral part of the fluoropolymer surface over no greater than 95% of the total area of the fluoropolymer surface, or no greater than 90, 85, or no greater than 80% of the total area of the fluoropolymer surface.

In some embodiments, the medical device comprises a tubular body comprising the fluoropolymer surface.

The fluoropolymer surface may be or comprise an outer and/or an inner surface of the tubular body. The fluoropolymer surface may preferably be or comprise an outer surface of the tubular body.

In some embodiments, the fluoropolymer surface comprises at least 5% of the outer surface area of the tubular body, or at least 10, 20, 30, 40, 50, 60, or preferably at least 70, or at least 80, 90, 95, 96, 97, 98, or at least 99% of the outer surface area of the tubular body, or 100% of the outer surface area of the tubular body. The fluoropolymer surface may comprise no greater than 95%, or no greater than 90, 85, or no greater than 80% of the outer surface area of the tubular body.

In some embodiments, the hyaluronic acid species is located at and/or on at least 50, 55, 60, 65, 70, 75, 80, 85, 90, 95, 96, 97, 98 or at least 99% of the outer surface area of the tubular body, preferably at least 75% or at least 90% of the outer surface area of the tubular body or between 75% and 100% of the outer surface area.

In preferred embodiments, the medical device is an insertable medical device. In some embodiments, the medical device is a cannula or a catheter, preferably which is configured to be inserted into a body. In preferred embodiments, at least one hyaluronic acid species is present at and/or on at least part of a surface of the cannula or catheter that is configured to be inserted into the body.

In some embodiments, the cannula or catheter is independently chosen from: a urinary cannula or catheter, an intravenous cannula or catheter, a nasal cannula or catheter, and a microcannula.

In some embodiments, the cannula or catheter is independently selected from the group consisting of: a urinary cannula or catheter, an intravenous cannula or catheter, a nasal cannula or catheter, and a microcannula.

The cannula or catheter may be an indwelling (Foley) catheter or cannula. Such a cannula/catheter is typically inserted and kept in a body for long periods of time, such as several days to months. Alternatively, the cannula or catheter may be an intermittent catheter or cannula. Such a cannula/catheter is typically inserted into a body for short time periods, such as less than a day.

In preferred embodiments, the medical device is a cannula that is part of an infusion set. The cannula may be part of an infusion set comprising a body which comprises a fluid part. In some embodiments, the body of the infusion set is attachable to the body of a user, in use. The body of the infusion set may be attachable to the body of the user via an adhesive part, in use. The adhesive part may be attachable to skin, in use. The adhesive part may attach the body of the infusion set to the user's skin, in use. The fluid part may be connected to the body of the infusion set or comprise part of the body of the infusion set. The fluid part may provide a fluid path through the infusion set. The fluid part may allow for fluid communication between the body of the infusion set and the cannula. The cannula may be attached to the fluid part or directly to the body of the infusion set. An end of the cannula may preferably be insertable into the body of a user, in use. In some embodiments, the cannula comprises an insertion needle on an end thereof, which can help to insert the cannula into the body of the user. The infusion set may further comprise an inserter part to assist insertion of the cannula into the body of the user. The inserter part may be an automatic inserter part or a manual inserter part.

The infusion set may further comprise a pump. The pump may assist in transporting substances from the infusion set into the body of a user, and vice versa. In some embodiments, the pump is attached to the insertion set via a connector. The pump may be attached to the body of the infusion set via the connector. The connector may comprise a tube which may be attached to a hub which controls the pump.

In some embodiments, the medical device is a cannula that is part of a patch pump. The patch pump may comprise a patch that is attachable to the body of a user, in use. The patch may comprise an adhesive. The patch may be attachable to skin through the adhesive, in use. The patch may comprise a fluid part. The fluid part may provide a fluid path through the patch pump. The cannula may be attached to the fluid part. An end of the cannula may preferably be insertable into the body of a user, in use. In some embodiments, the cannula comprises an insertion needle on an end thereof, which can help to insert the cannula into the body of the user. The patch may further comprise a pump, which may be an integral part of the patch or may be attached thereto. The pump may assist in transporting substances from the patch pump into the body of a user, and vice versa.

In some preferred embodiments, there is provided a medical device comprising a fluoropolymer surface comprising at least one hyaluronic acid species, wherein the medical device is a cannula that is part of an infusion set or patch pump.

In some embodiments, the cannula is part of an infusion set or patch pump for the delivery of a substance into the body. The cannula may be part of an intravenous and/or subcutaneous infusion set or patch pump. The cannula may be part of an infusion set or patch pump for the subcutaneous delivery of a substance into the body, such as for the subcutaneous delivery of insulin into the body.

In some embodiments, the catheter or cannula comprises a hollow tubular body. The hollow tubular body may comprise an outer surface and/or an inner surface. The outer surface may comprise at least one chosen from: an external facing surface of the body, a lumen of the body, and any eyelets present on the body. The outer surface may comprise at least one of the group consisting of: an external facing surface of the body, a lumen of the body, and any eyelets present on the body. In preferred embodiments, the outer surface is the external-facing surface of the body and/or the inner lumen. In some embodiments, the outer surface may comprise the external-facing surface of the body, the inner lumen, and the eyelets. The inner surface of the body may comprise a lumen of the body.

In some embodiments, the hyaluronic acid species is present at and/or on an inner surface of the body, an outer surface of the body, or both. In preferred embodiments, the hyaluronic acid species is present at and/or on at least an outer surface of the body.

According to the second aspect of the invention, there is provided an infusion set or patch pump comprising a cannula comprising a fluoropolymer surface comprising at least one hyaluronic acid species.

The cannula may preferably be a medical device of the first aspect of the invention. Statements of invention for the first aspect of the invention above may also be applied mutatis mutandis to the second aspect of the invention.

According to a third aspect of the invention, there is provided a method of manufacturing a medical device, the method comprising the steps of:
(a) Providing a medical device comprising a fluoropolymer surface;
(b) Activating the fluoropolymer surface; and
(c) Functionalising the activated fluoropolymer surface with at least one hyaluronic acid species.

The medical device of the third aspect of the invention is preferably the medical device of the first aspect of the invention. Statements of invention above relating to the medical device of the first aspect of the invention or to any of its components may also be applied to the third aspect of the invention. Other statements of invention for the first and second aspects of the invention above may also be applied mutatis mutandis to the third aspect of the invention.

Statements of invention below relating to the third aspect of the invention may also be applied mutatis mutandis to the first and second aspects of the invention.

In some embodiments, step (a) comprises forming the medical device by a melt-extrusion or injection moulding procedure. The method may comprise melt-extruding or injection moulding a fluoropolymer to form a tubular body of the medical device. In some embodiments, the fluoropolymer is provided in granulate or powder form prior to melt-extrusion or injection-moulding.

In some embodiments, step (b) comprises introducing at least one reactive group on the fluoropolymer surface. Step (b) may comprise cleaving at least one polymer chain on the fluoropolymer surface, and introducing at least one reactive group on the surface. In some embodiments, at least one reactive group comprises at least one electronegative atom. In some embodiments, at least one reactive group may be independently chosen from: an oxygen-containing moiety, an unsaturated moiety, a radical, and combinations thereof. In some embodiments, at least one reactive group may be independently selected from the group consisting of: an oxygen-containing moiety, an unsaturated moiety, a radical, and combinations thereof.

Step (b) may comprise oxidising the fluoropolymer surface. In some embodiments, step (b) is performed under atmospheric oxygen conditions. In other embodiments, step (b) may be performed under an oxygen enriched atmosphere. Step (b) may produce an activated fluoropolymer surface comprising at least one oxygen-containing reactive moiety. At least one oxygen-containing moiety may be independently chosen from: a peroxy group, a hydroxy group, a carbonyl group, and derivatives and/or combinations thereof. At least one oxygen-containing moiety may be independently selected from the group consisting of: a peroxy group, a hydroxy group, a carbonyl group, and derivatives and/or combinations thereof. The carbonyl group may be independently chosen from: a carboxyl group, an aldehyde, a ketone, an acid fluoride, and combinations thereof. The carbonyl group may be independently selected from the group consisting of: a carboxyl group, an aldehyde, a ketone, an acid fluoride, and combinations thereof.

In some embodiments, step (b) comprises producing an activated fluoropolymer surface comprising at least one unsaturated reactive moiety. At least one unsaturated reactive moiety may be independently chosen from: an alkene, an alkyne, and derivatives and/or combinations thereof. At least one unsaturated reactive moiety may be independently selected from the group consisting of: an alkene, an alkyne, and derivatives and/or combinations thereof. Such unsaturated reactive moieties may react via polymerisation-type reactions. In any of the embodiments described herein in which polymerisation is performed, any suitable polymerisation process may be used, such as conventional condensation, addition or free radical graft polymerization (FRGP) or controlled radical polymerization (CRP), such as ATRGP, RAFT and NMGP.

In some embodiments, step (b) comprises the step of activating the fluoropolymer surface across at least 5% of the total area of the fluoropolymer surface, or at least 10, 20, 30, 40, 50, 60, 70, 80, 90, 95, 96, 97, 98, or across at least 99% of the total area of the fluoropolymer surface, or 100% of the total area of the fluoropolymer surface. Step (b) may comprise the step of activating the fluoropolymer surface across no greater than 95% of the total area of the fluoropolymer surface, or across no greater than 90, 85, or no greater than 80% of the total area of the fluoropolymer surface.

Step (b) may comprise defluorinating or partially defluorinating the fluoropolymer surface. Step (b) may comprise defluorinating at least 5% of the fluoropolymer surface, or at least 10, 20, 30, 40, 50, 60, 70, 80, 90, 95, 96, 97, 98, or at least 99% of the fluoropolymer surface, or 100% of the fluoropolymer surface. Step (b) may comprise defluorinating no greater than 95% of the fluoropolymer surface, or no greater than 90, 85, or no greater than 80% of the fluoropolymer surface.

Step (b) may comprise reducing the average fluorine-to-carbon atomic ratio (F/C ratio) of the fluoropolymer surface to a value of no greater than 1.2, or no greater than 1, 0.9, 0.8, 0.7, 0.6, 0.5, 0.4, 0.3, 0.2, or no greater than 0.1.

Step (b) may comprise increasing the average surface energy of the fluoropolymer surface to a value of at least 25 mN/m, or at least 30, 35, 40, 45, 50, 55, 60, or at least 65 mN/m.

Step (b) may comprise reducing the average contact angle of the fluoropolymer surface to a value of no greater than 80°, or no greater than 70, 60, 50, 40, or no greater than 30°.

Step (b) may comprise activating the fluoropolymer surface with at least one fluoropolymer surface activation method independently chosen from: plasma treatment, treatment with a reducing agent, corona discharge treatment, ion beam treatment, laser treatment, and combinations thereof.

Step (b) may comprise activating the fluoropolymer surface with at least one fluoropolymer surface activation method independently selected from the group consisting of: plasma treatment, treatment with a reducing agent, corona discharge treatment, ion beam treatment, laser treatment, and combinations thereof.

In some embodiments, step (b) comprises the step of plasma treating the fluoropolymer surface.

In some preferred embodiments, the method of manufacturing a medical device comprises the steps of:
(a) Providing a medical device comprising a fluoropolymer surface;
(b) Activating the fluoropolymer surface by plasma treating the fluoropolymer surface; and
(c) Functionalising the activated fluoropolymer surface with at least one hyaluronic acid species.

Plasma treating the fluoropolymer surface may comprise applying a plasma stream to the fluoropolymer surface. The fluoropolymer surface may be directly contacted with plasma as it is generated, or in a separate post-plasma area. If the surface is directly contacted with plasma during generation, this may take place in a plasma reactor. By post-plasma area, it is meant in the present disclosure an area out of the plasma, located downstream of a plasma forming gas flow introduced in the plasma wherein reactive species such as radicals are still present. That post-plasma area is particularly useful for delicate substrate surfaces such as polymers.

Step (b) may comprise treating the fluoropolymer surface with a gaseous plasma. The plasma may comprise at least one plasma gas independently chosen from: hydrogen, oxygen, nitrogen, air, ammonia, argon, helium, carbon dioxide, water, methane, ethane, propane, butane, and any mixture thereof. The plasma may comprise at least one plasma gas independently selected from the group consisting of: hydrogen, oxygen, nitrogen, air, ammonia, argon, helium, carbon dioxide, water, methane, ethane, propane, butane, and any mixture thereof. The plasma gas may be carried by a carrier gas, which may be the same as the plasma gas or may be different to the plasma gas. In some embodiments, the carrier gas is an inert gas, such as argon, for example.

In some embodiments, the method may comprise treating the fluoropolymer surface with a primary gas and a secondary gas. The primary gas may be independently chosen from: hydrogen, oxygen, nitrogen, air, ammonia, argon, helium, carbon dioxide, water, methane, ethane, propane, butane, and any mixture thereof. The primary gas may be independently selected from the group consisting of: hydrogen, oxygen, nitrogen, air, ammonia, argon, helium, carbon dioxide, water, methane, ethane, propane, butane, and any mixture thereof. The primary gas may comprise an inert gas, which may comprise a noble gas. The primary gas may be independently chosen from: helium, argon, and combinations thereof. The primary gas may be independently selected from the group consisting of: helium, argon, and combinations thereof. The secondary gas may be independently chosen from: hydrogen, oxygen, nitrogen, air, ammonia, argon, helium, carbon dioxide, water, methane, ethane, propane, butane, and any mixture thereof. The secondary gas may be independently selected from the group consisting of: hydrogen, oxygen, nitrogen, air, ammonia, argon, helium, carbon dioxide, water, methane, ethane, propane, butane, and any mixture thereof. In some embodiments, the secondary gas is or comprises oxygen.

In some embodiments, the method may comprise treating the fluoropolymer surface with at least one plasma gas having a flow rate of at least 3 Lpm, or at least 6, 9, 12, or at least 15 Lpm. The method may comprise treating the fluoropolymer surface with at least one plasma gas having a flow rate of no greater than 50 Lpm, or no greater than 45, 40, 35, 30, 25, or of no greater than 20 Lpm. The method may comprise treating the fluoropolymer surface with at least one plasma gas having a flow rate of between 5-30 Lpm, or between 10-25, or between 15-20 Lpm. At least one plasma gas having such a flow rate may preferably be a primary gas.

In some embodiments, the method may comprise treating the fluoropolymer surface with at least one plasma gas having a flow rate of at least 0.025 Lpm, or at least 0.05, 0.075, 0.1, 0.2, 0.3, 0.4, 0.5, or at least 0.6 Lpm. The method may comprise treating the fluoropolymer surface with at least one plasma gas having a flow rate of no greater than 5 Lpm, or no greater than 4, 3, 2, 1, 0.9, 0.8, or no greater than 0.7 Lpm. The method may comprise treating the fluoropolymer surface with at least one plasma gas having a flow rate of between 0.025-1 Lpm, or of between 0.05-0.9, 0.075-0.8, 0.1-0.7, or between 0.15-0.65 Lpm. At least one plasma gas having such a flow rate may preferably be a secondary gas.

The step of plasma treating the fluoropolymer surface may introduce at least one reactive group on the fluoropolymer surface, preferably at least one oxygen-containing reactive moiety. Plasma treating the fluoropolymer surface may oxidise the fluoropolymer surface. In some embodiments, plasma treating the fluoropolymer surface is performed under atmospheric oxygen conditions. In other embodiments, plasma treating the fluoropolymer surface may be performed under an oxygen enriched atmosphere.

In some embodiments, the plasma treatment step uses cold plasma.

Cold plasma, otherwise known as non-thermal or non-equilibrium plasma, is the term used for cold temperature plasma formation at atmospheric pressures. Cold plasma is a plasma which is not in thermodynamic equilibrium, because the electron temperature is much hotter than the temperature of heavy species (ions and neutrals) in the plasma. Cold plasma is created when a sufficient amount of energy, higher than the ionization energy, is added to gaseous atoms and/or molecules, causing ionization and subsequently generating free electrons, photons, free radicals and ionic species. This excitation energy supplied to a gas to form a cold plasma can originate from electrical discharges, direct currents, radio frequencies, microwaves or other forms of electromagnetic radiation.

Non-limiting examples of cold plasma technologies and methodologies for generating cold plasma include atmospheric pressure plasma jet, dielectric barrier discharge, direct current (DC) glow discharge, electrical discharge plasma, microwave discharge, pulsed power discharge, radiofrequency (RF) discharge, and the like.

In some embodiments, the cold plasma is cold atmospheric plasma. The cold plasma may be an atmospheric pressure discharge cold plasma.

The temperature of the cold plasma may be at least 5° C. or at least 10° C. The temperature of the cold plasma may be no more than 60° or no more than 50° C. In some embodiments the cold plasma is at ambient temperature, such as between 15° C. and 35° C., for example.

The plasma or cold plasma may be at a pressure of between around 50 kPa and 150 kPa, preferably between around 60 kPa and 140 kPa, between around 70 kPa and 130 kPa, or between around 80 kPa and 120 kPa. In some embodiments the pressure may be between around 100 kPa and 103 kPa.

In other embodiments, the plasma or cold plasma may be applied under reduced pressure such as below 50 kPa, such as between 0.01 kPa and 40 kPa, or between 0.1 kPa and 25 kPa.

The plasma or cold plasma treatment may be performed at a radio-frequency (RF) power of at least 1 W, 5 W, 10 W, 15 W or at least 20 W. The plasma or cold plasma treatment may be performed at an RF power of no more than 2000 W, 1500 W, 1000 W, 500 W, 400 W, 300 W, 200 W, 100 W, 90 W, 80 W, 70 W or no more than 60 W. In some embodiments the treatment may be performed at an RF power of about 20 to 60 W. The treatment may be performed at an RF power of between 20-500 W, or between 30-450, 40-400, 50-350, 60-300, 70-250, 80-200, or between 90-170, or between 100-160 W.

The plasma or cold plasma treatment may be performed for a total time of at least 1 second, 2 seconds, 3 seconds, 4 seconds, 5 seconds or at least 10 seconds. The plasma or cold plasma treatment may be performed for no more than 600 seconds, 550 seconds, 500, 450, 400, or no more than 350 seconds. In some embodiments, the treatment may be performed for about 5 to 500 seconds, or for about 10-400 seconds, or for about 15-300 seconds.

The plasma or cold plasma treatment may be performed at a temperature of at least 5° C., or at least 10, 20, 30, 40, 50, or at least 60° C. The plasma or cold plasma treatment may be performed at a temperature of no greater than 200° C., or no greater than 180, 160, 140, 120, 100, 80, or no greater than 60° C. The plasma or cold plasma treatment may be performed at a temperature of between 20-100° C., or between 30-90, 40-80, 50-70, or between 55-65° C.

The plasma or cold plasma treatment may be performed at an RF power of between about 10 W to about 60 W, for a period of between about 5 seconds to about 120 seconds; and in some embodiments may be performed using the aforesaid RF and time ranges using a precursor gas selected from the group consisting of hydrogen, oxygen, nitrogen, argon or helium.

In some embodiments, step (b) comprises treating the fluoropolymer surface with at least one reducing agent.

In some preferred embodiments, the method of manufacturing a medical device comprises the steps of:
(a) Providing a medical device comprising a fluoropolymer surface;
(b) Activating the fluoropolymer surface by treating the fluoropolymer surface with at least one reducing agent; and
(c) Functionalising the activated fluoropolymer surface with at least one hyaluronic acid species.

The reducing agent may act to transfer electrons to the fluoropolymer surface. Such a method is particularly effective at producing a highly reactive fluoropolymer surface which can be easily functionalised with a hyaluronic acid species. However, the method has no long-term implications on the stability of the modified surface—the method may in fact aid stability of the modified surface through surface crosslinking interactions generated on treatment with a reducing agent.

Treating the fluoropolymer surface with at least one reducing agent may generate at least one surface reactive group. In some embodiments, at least one reactive group may be as described in statements above and may be independently chosen from: an oxygen-containing moiety, an unsaturated moiety, a radical, and combinations thereof. In some embodiments, at least one reactive group may be as described in statements above and may be independently selected from the group consisting of: an oxygen-containing moiety, an unsaturated moiety, a radical, and combinations thereof.

The method may comprise the steps of: transferring at least one electron from the reducing agent to the fluoropolymer surface to generate a negatively charged or partially negatively charged surface group; and removing at least one fluorine from the surface group to generate a neutral defluorinated surface group. Fluorine may be removed from the surface group as fluoride or a derivative thereof. In some embodiments, the above steps may produce a radical-containing neutral defluorinated surface group. The above steps may be repeated to generate a non-radical neutral defluorinated surface group. The non-radical neutral defluorinated surface group may comprise a reactive group, preferably an unsaturated moiety, such as an alkene. The reactive group may participate in step (c) of the method of the third aspect of the invention. In some embodiments, the above steps may not be repeated, and the radical-containing neutral defluorinated surface group may participate directly in step (c) of the method of the third aspect of the invention.

At least one reducing agent used in step (b) of the invention may be independently chosen from: an alkali metal, an alkaline earth metal, a group III metal, a transition metal, and combinations thereof.

At least one reducing agent used in step (b) of the invention may be independently selected from the group consisting of: an alkali metal, an alkaline earth metal, a group III metal, a transition metal, and combinations thereof.

In preferred embodiments, at least one reducing agent comprises an alkali metal and/or an alkaline earth metal. At least one reducing agent may preferably comprise an alkali metal. At least one reducing agent may comprise an alkali metal independently chosen from: lithium, potassium, sodium, and combinations thereof. At least one reducing agent may comprise an alkali metal independently selected from the group consisting of: lithium, potassium, sodium, and combinations thereof. In a particularly preferred embodiment, at least one reducing agent comprises sodium.

In some embodiments, at least one reducing agent may be used with a stabilising species. The stabilising species may complex the reducing agent, preferably in the form of a salt. The stabilising species may accept an electron from the reducing agent, preferably to form a radical anion. The stabilising species may preferably be an aromatic compound. The stabilising species may be a polycyclic aromatic compound. The stabilising species may be independently chosen from: benzene, naphthalene, biphenyl, anthracene, pyrene, acenaphthylene, perylene, and derivatives thereof. The stabilising species may be independently selected from the group consisting of: benzene, naphthalene, biphenyl, anthracene, pyrene, acenaphthylene, perylene, and derivatives thereof. The stabilising species may preferably be naphthalene or a derivative thereof. In preferred embodiments, at least one reducing agent comprises an alkali metal and a naphthalene stabilising species which forms an alkali metal naphthalide, preferably sodium naphthalide.

At least one reducing agent may be provided as a solution. At least one reducing agent may be dissolved in a carrier solvent to provide the solution. The carrier solvent may comprise an aprotic solvent. The carrier solvent may comprise an ether, preferably an aprotic ether. In some embodiments, the carrier solvent comprises a glycol ether, preferably an aprotic glycol ether, such as a dialkyl glycol ether. In preferred embodiments, the carrier solvent is independently chosen from: monoglyme, diglyme, tetraglyme, and combinations thereof. In preferred embodiments, the carrier solvent is independently selected from the group consisting of: monoglyme, diglyme, tetraglyme, and combinations thereof. In a particularly preferred embodiment, the carrier solvent comprises diglyme.

Particularly preferably, the reducing agent comprises an alkali metal, preferably sodium and the carrier solvent comprises an aprotic glycol ether, preferably a dialkyl glycol ether, more preferably diglyme.

Such solvents enable high temperature etching, which accelerates and reduces the length of the surface treatment process.

In some embodiments, step (b) comprises treating the fluoropolymer surface with at least one reducing agent at a temperature of at least 5° C., or at least 10, 15, 20, 25, 30, 35, 40, or at least 45° C. Step (b) may comprise treating the fluoropolymer surface with at least one reducing agent at a temperature of no greater than 500° C., or no greater than 450, 400, 350, 300, 250, 200, 150, 100, 90, 80, 70, 60, or no greater than 50° C. Step (b) may comprise treating the fluoropolymer surface with at least one reducing agent at a temperature of between 5-100° C., or between 10-95, 20-90, 25-85, 30-80, 35-75, 40-70, 45-65, or between 50-65° C. Step (b) may comprise treating the fluoropolymer surface with at least one reducing agent at a temperature of between 10-70° C., or between 15-65, or between 20-60° C.

Such temperatures allow more active reducing agent to be released. Reducing agent viscosity is also reduced which allows for wetting of high aspect ratio features of the medical device.

In some embodiments, step (b) comprises treating the fluoropolymer surface with at least one reducing agent at a temperature of between 30-80° C., or between 35-75, 40-70, 45-65, or between 50-60° C.; and wherein the reducing agent is dissolved in a glycol ether carrier solvent, preferably an aprotic glycol ether solvent, more preferably a dialkyl glycol ether.

Step (b) may comprise treating the fluoropolymer surface with the reducing agent for at least 1 second, or at least 2, 3, 4, 5, 10, 15, 20, 25, or at least 30 seconds. Step (b) may comprise treating the fluoropolymer surface with the reducing agent for no greater than 300 seconds, or no greater than 280, 260, 240, 220, 200, 180, 160, 140, 120, 100, 80, or no greater than 60 seconds. Step (b) may comprise treating the fluoropolymer surface with the reducing agent for between 5-180 seconds, or between 10-160, 15-140, 20-120, 25-110, 30-100, 35-90, 40-80, 50-70, or between 55-65 seconds. Step (b) may comprise treating the fluoropolymer surface with the reducing agent for between 5-55 seconds, or between 10-50, 15-45, 20-40, or between 25-35 seconds.

Step (b) may comprise applying the reducing agent to the fluoropolymer surface, preferably as a solution. Step (b) may comprise submerging the medical device or the fluoropolymer surface in the solution.

Step (c) may comprise treating the activated fluoropolymer surface with at least one hyaluronic acid species, preferably as described for the first aspect of the invention.

Step (c) may comprise treating the surface with a solution of the hyaluronic acid species in a solvent. The solvent may be a polar solvent, preferably a polar protic solvent. Particularly preferably, the solution may be an aqueous solution. The solvent may be or comprise water. Alternatively, the solution may comprise an organic solvent, which may be a polar organic solvent. The organic solvent may be independently chosen from: an alcohol, an ether, an ester, a ketone, an aldehyde, an amide, a nitrile, a sulfoxide, a carbonate, a carboxylic acid, and combinations thereof. The organic solvent may be independently selected from the group consisting of: an alcohol, an ether, an ester, a ketone, an aldehyde, an amide, a nitrile, a sulfoxide, a carbonate, a carboxylic acid, and combinations thereof. The hyaluronic acid species may be present in the solution at a total concentration of at least 0.05 wt. %, or at least 0.1, 0.2, 0.3, 0.4, 0.5, 0.6, 0.7, 0.8, 0.9, or at least 1 wt. %. The hyaluronic acid species may be present in the solution at a total concentration of no greater than 10 wt. %, or no greater than 9, 8, 7, 6, 5, 4, 3, 2, or no greater than 1 wt. %. The hyaluronic acid species may be present in the solution at a total concentration of between 0.05-5 wt %, or between 0.1-2 wt. %, or between 0.5-1.5, or between 0.75-1.25 wt. %.

Step (c) may comprise treating the surface with at least one hyaluronic acid species for a total time of at least 5 minutes, or at least 10, 20, 30, 40, 50, 60, 70, 80, 90, 100, 110, 120, 130, 140, 150, 160, 170, 180, 190, 200, 210, 220, 230, or at least 240 minutes. Step (c) may comprise treating the surface with at least one hyaluronic acid species for total time of no greater than 10 hours, or no greater than 9, 8, 7, 6, 5, 4.5, or no greater than 4 hours. Step (c) may comprise treating the surface with at least one hyaluronic acid species for total time of between 0.5-7.5 hours, or between 1-7, 1.5-6.5, 2-6, 2.5-5.5, 3-5, or between 3.5-4.5 hours.

Step (c) may comprise treating the surface with at least one hyaluronic acid species at a temperature of at least 5° C. or at least 10, 15 or at least 20° C. Step (c) may comprise treating the surface with at least one hyaluronic acid species at a temperature of no greater than 100° C., or no greater than 90, 80, 70, 60, 50, 40, or no greater than 30° C. Step (c) may comprise treating the surface with at least one hyaluronic acid species at a temperature of between 5-45° C., or between 10-40, 15-35, or between 20-30° C.

Step (c) may comprise grafting at least one polymeric hyaluronic acid species to the activated surface. The method may alternatively comprise polymerising at least one hyaluronic acid species from the activated surface, preferably by attaching a hyaluronic acid species monomer to the surface and polymerising from the monomer to provide a polymeric hyaluronic species attached to the fluoropolymer surface.

The step of functionalising the activated fluoropolymer surface with at least one hyaluronic acid species may comprise bonding the hyaluronic acid to the activated surface through a linker. The linker may be derived from a linking compound, preferably as described for the first aspect of the invention. The method may comprise the further step of treating the activated fluoropolymer surface with a linking compound. The method may comprise the step of first bonding the linking compound to the activated fluoropolymer surface, and then bonding at least one hyaluronic acid species to the linking compound. The method may comprise the step of treating the activated surface with the linking compound, optionally in the absence or presence of the hyaluronic acid species; and then treating the surface with at least one hyaluronic acid species. In some embodiments, the method comprises functionalising the activated surface with the linking compound to form a layer of the linking compound attached to the fluoropolymer surface.

The method may comprise treating the fluoropolymer surface with the linking compound for a total time of at least 5 minutes, or at least 10, 20, 30, 40, 50, or at least 60 minutes. The method may comprise treating the surface with the linking compound for a total time of no greater than 300 minutes, or no greater than 250, 200, or no greater than 150 minutes. The method may comprise treating the surface with the linking compound for a total time of between 20-100 minutes, or between 30-90, 40-80, 50-70, or between 55-65 minutes.

The method may comprise treating the surface with the linking compound at a temperature of at least 5° C. or at least 10, 15 or at least 20° C. The method may comprise treating the surface with the linking compound at a temperature of no greater than 100° C., or no greater than 90, 80, 70, 60, 50, 40, or no greater than 30° C. The method may comprise treating the surface with the linking compound at a temperature of between 5-45° C., or between 10-40, 15-35, or between 20-30° C.

The linking compound may be present neat or as a solution of the linking compound in a solvent. The solvent may be a polar solvent, preferably a polar protic solvent. In some embodiments, the solution may be an aqueous solution. The solvent may be or comprise water. Alternatively, the solution may comprise an organic solvent, which may be a polar organic solvent. The organic solvent may be independently chosen from: an alcohol, an ether, an ester, a ketone, an aldehyde, an amide, a nitrile, a sulfoxide, a carbonate, a carboxylic acid, and combinations thereof. The organic solvent may be independently selected from the group consisting of: an alcohol, an ether, an ester, a ketone, an aldehyde, an amide, a nitrile, a sulfoxide, a carbonate, a carboxylic acid, and combinations thereof. The linking compound may be present in the solution at a total concentration of at least 0.05 wt. %, or at least 0.1, 0.2, 0.3, 0.4, 0.5, 0.6, 0.7, 0.8, 0.9, or at least 1 wt. %. The linking compound may be present in the solution at a total concentration of no greater than 10 wt. %, or no greater than 9, 8, 7, 6, 5, 4, 3, 2, or no greater than 1 wt. %. The linking compound may be present in the solution at a total concentration of between 0.05-5 wt %, or between 0.1-2 wt. %, or between 0.5-1.5, or between 0.75-1.25 wt. %.

At least one hyaluronic acid species or monomer thereof may be provided in a free acid or salt form. The salt may comprise a carboxylate anion and a cation. The cation may be independently chosen from: an alkali metal, such as sodium or potassium; an alkaline earth metal; and a nitrogen-containing cation, such as ammonium, substituted ammonium, and quaternized derivatives thereof, such as tetraalkylammonium (e.g. tetrabutylammonium). The cation may be independently selected from the group consisting of: an alkali metal, such as sodium or potassium; an alkaline earth metal; and a nitrogen-containing cation, such as ammonium, substituted ammonium, and quaternized derivatives thereof, such as tetraalkylammonium (e.g. tetrabutylammonium). The nitrogen-containing cation may comprise an aromatic nitrogen cation, such as pyridinium or a derivative thereof (e.g. collidinium).

At least one hyaluronic acid species or monomer thereof may be provided as a chemically modified derivative of the hyaluronic acid species. The chemically modified derivative may comprise at least one reactive group, preferably to facilitate attachment of the hyaluronic acid species to the fluoropolymer surface or linking compound. The reactive group may comprise a polymerizable moiety, preferably an unsaturated moiety. The unsaturated moiety may preferably comprise an acrylate or methacrylate moiety. In some embodiments, the method comprises polymerising at least one hyaluronic acid species through the polymerizable moiety. The method may comprise polymerising a polymeric hyaluronic acid species to produce a polymer comprising hyaluronic acid specie macromonomers.

In any of the embodiments described herein in which polymerisation is performed, any suitable polymerisation process may be used, such as conventional condensation, addition or free radical graft polymerization (FRGP) or controlled radical polymerization (CRP), such as ATRGP, RAFT and NMGP.

In some embodiments, steps (b) and (c) are performed simultaneously. In other embodiments, step (c) may be performed subsequently to step (b).

In some preferred embodiments, the method of manufacturing a medical device comprises the steps of:
(a) Providing a medical device comprising a fluoropolymer surface;
(b) Activating the fluoropolymer surface; and
(c) Functionalising the activated fluoropolymer surface with at least one hyaluronic acid species,
wherein steps (b) and (c) are performed simultaneously.

In some preferred embodiments, the method of manufacturing a medical device comprises in order the steps of:
(a) Providing a medical device comprising a fluoropolymer surface;
(b) Activating the fluoropolymer surface; and then
(c) Functionalising the activated fluoropolymer surface with at least one hyaluronic acid species.

In some embodiments, the activation step is performed in the presence of at least one hyaluronic acid species. In other embodiments, the activation step is performed in the absence of the hyaluronic acid species, preferably prior to addition of the hyaluronic acid species.

In embodiments in which at least one hyaluronic acid species is bonded to the fluoropolymer surface through a linker, the method may comprise the step of functionalising the fluoropolymer surface with the linking compound simultaneously or subsequently to step (b). In some embodiments, the activation step is performed in the presence of the linking compound. In other embodiments, the activation step is performed in the absence of the linking compound, preferably prior to addition of the linking compound.

In embodiments in which step (b) comprises plasma treating the fluoropolymer surface, the method may comprise the step of subjecting the fluoropolymer surface to a first plasma jet in the presence of a linking compound to form a layer of the linking compound on the fluoropolymer surface. The method may comprise the further step of subjecting the linking compound layer to a second plasma jet in the presence of at least one hyaluronic acid species to attach the hyaluronic acid species to the linking compound layer.

In some embodiments, the method comprises a further step of sonicating the fluoropolymer surface. The sonication step may be performed at one or more of the following times: after step (b), after treating the fluoropolymer surface with a linking compound and before treating the surface with at least one hyaluronic acid species, and at the end of step (c), and any combination thereof. The or each sonication step may be performed for between 1-30 minutes, or between 5-20 minutes, or between 5-15 minutes. The sonication step may be performed in a polar solvent, which may be a polar protic solvent. The solvent may be an aqueous solvent and may be water.

The method may comprise a further step of washing the fluoropolymer surface. The washing step may be performed at one or more of the following times: after step (b), after treating the fluoropolymer surface with a linking compound and before treating the surface with at least one hyaluronic acid species, and at the end of step (c), and any combination thereof. The surface may be washed with a solvent, which may be a polar solvent. The solvent may be a polar protic solvent. The solvent may comprise an alcohol and/or water. The washing step may be performed at a temperature of between 20-120° C., or between 40-100, or between 60-80° C. The washing step may comprise a first washing step at ambient temperature and a second washing step at a temperature range independently selected from the above range. The first washing step may be performed with an organic solvent, preferably a polar organic solvent. The polar organic solvent may comprise a polar protic solvent, such as an alcohol. The second step may be performed with an aqueous solution or with water, preferably with deionised water.

According to a fourth aspect of the invention, there is provided the use of a hyaluronic acid species as a protein-repellent in and/or on a medical device.

According to a fifth aspect of the invention, there is provided a method of delivering a substance to or removing a substance from the body of a subject, the method comprising the steps of:
(a) Inserting a medical device comprising a fluoropolymer surface comprising at least one hyaluronic acid species into the body; and
(b) Delivering a substance to or removing a substance from the body via the medical device.

The medical device may be a catheter or cannula, preferably as described for the first aspect of the invention. The medical device may be a cannula that is part of an infusion set or patch pump.

The method may comprise inserting the medical device into the body intravenously and/or subcutaneously.

The substance may be a drug. In some embodiments, the substance comprises insulin. Step (b) of the method may comprise delivering insulin to the body via the medical device.

The following statements apply to the fourth and fifth aspects of the invention.

The hyaluronic acid species and/or medical device may preferably be the hyaluronic acid species and medical device of the first aspect of the invention. Statements of invention for the first, second and third aspects of the invention above may also be applied mutatis mutandis to the fourth and fifth aspects of the invention.

### DETAILED DESCRIPTION OF THE INVENTION

In order that the invention may be more clearly understood embodiments thereof will now be described, by way of example only, with reference to the accompanying drawings, of which:

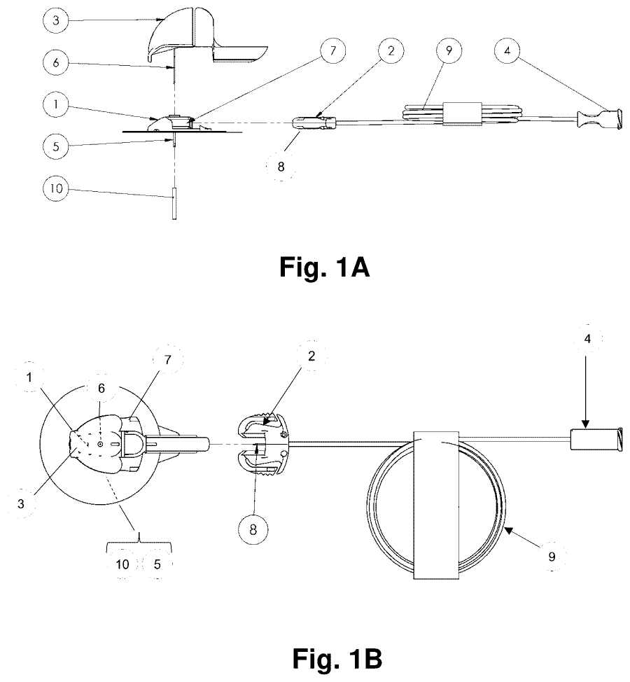

FIG. 1A shows an exploded side-on view.

FIG. 1B shows a top-down view of an infusion set of the second aspect of the invention. Dashed lines represent points of connection of the components of the infusion set.

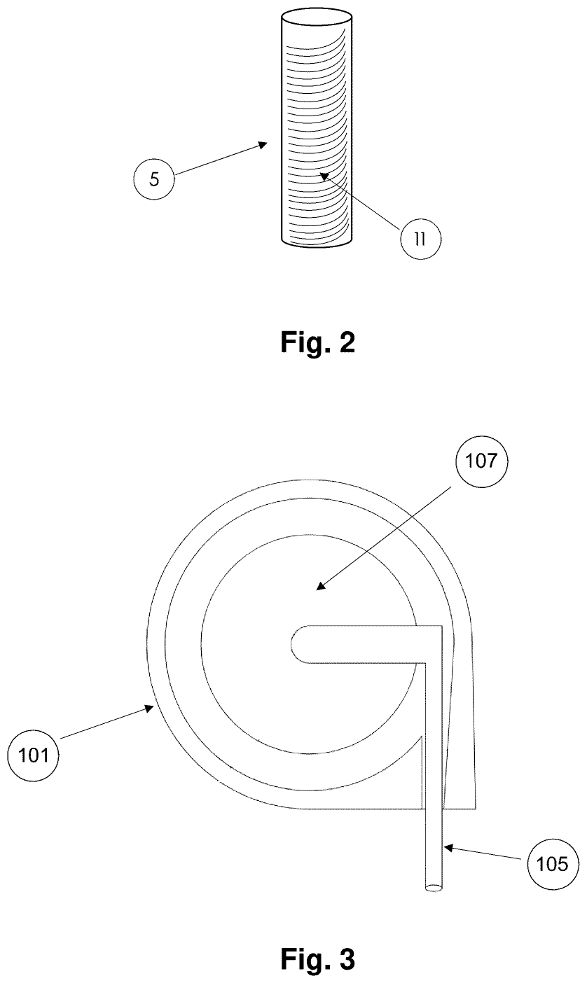

FIG. 2 shows an expanded side-on view of the cannula (5) as displayed in FIG. 1A.

FIG. 3 shows a cross-sectional view of a patch pump of the second aspect of the invention.

### EXAMPLE 1

A first embodiment of a medical device of the first aspect of the invention is provided by a cannula containing a polymeric tubular body having a PTFE outer surface. The PTFE outer surface is functionalised with hyaluronan, which is bonded to the PTFE surface via a linker derived from epichlorohydrin.

The cannula is part of an infusion set of the second aspect of the invention for the subcutaneous delivery of insulin. Diagrams of the infusion set are displayed in FIGS. 1A and 1B. With reference to the figures, the infusion set comprises a body (1), which is attachable to the skin of a user via an adhesive part of the body (1). The infusion set comprises the cannula (5) which extends from and projects away from the body (1) of the infusion set in the same direction that the adhesive part of the body (1) faces.

The body (1) comprises a fluid part (7), which is part of the body and provides a fluid path through the infusion set, allowing for fluid communication between the body (1) and the cannula (5). The fluid part (7) also contains a cartridge of insulin (not shown) for subcutaneous delivery. The arrangement also allows for fluid communication between the inside of the insulin cartridge and the cannula (5). The fluid part (7) is connected to a pump (not shown) via tubing (9). The fluid part (7) is connected to the tubing (9) at one end thereof via a connector needle (8) of a set connector (2). The other end of the tubing (9) contains a pump connector (4) through which the tubing (9) is attached to the pump.

The fluid part (7) and body (1) contain a channel extending therethrough which is aligned with the cannula (5). Such an arrangement allows for an insertion needle (6) to be passed through the channel and into the cannula (5), with the insertion needle (6) projecting in the same direction as the cannula (5) and extending out of the free, distal end of the cannula (5). An inserter (3) is connected to the insertion needle (6) and the needle (6) extends from the inserter (3). The infusion set further includes a needle cover (10) in which the insertion needle (6) is sheathed before use.

In use, the body (1) of the infusion set is attached to the skin of the user via the adhesive part of the body (1). The free end of the cannula (5), which projects from the body (1), is inserted into the body of the user with assistance from the insertion needle (6), which is inserted using the inserter (3) through the channel extending through the fluid part (7) and body (1) and through the cannula (5). The insertion needle (6) contacts the skin of the user and is inserted into the body of the user before the cannula (5), making insertion of the cannula (5) easier.

On insertion of the cannula (5) into the body of the user, the fluid part (7) is connected to the pump as described above. The insulin is delivered subcutaneously from the infusion set via the cannula (5), with assistance from the pump.

FIG. 2 shows an expanded side-on view of the cannula (5), as displayed in FIG. 1A. FIG. 2 displays the hyaluronic acid species as a layer or coating (11), which is formed on the PTFE outer surface of the cannula by bonding of the species to the PTFE.

### Cannula Preparation Method

The functionalised cannula was prepared as follows.

Surface Activation: a solution of sodium naphthalide in diglyme was preheated to 60° C. for 1 hour. The solution was then shaken vigorously for 2-3 seconds, after which the cannula was submerged in the solution for 30 seconds. The cannula was thereafter removed and immediately rinsed with isopropyl alcohol for 10 seconds. The cannula was then further rinsed with 70° C. deionised water for 15 seconds. The cannula was then left to air dry overnight.

XPS data showed that the fluorine-to-carbon atom ratio had changed from 2:1 (before surface activation) to 1:6 (after activation). XPS data also showed the presence of C—O bonds on the treated surface.

Linker functionalisation: the cannula was thereafter submerged in neat epichlorohydrin at 50° C. for 2 hours. The cannula was then removed, rinsed with deionised water at ambient temperature, and sonicated for 10 minutes in fresh deionised water. The cannula was then air dried.

Hyaluronic acid functionalisation: a 1 wt. % solution of sodium hyaluronate (MWt 1.5-2.2 MDa) in deionised water was prepared. The cannula was submerged in the prepared solution at room temperature for 2 hours, prior to rinsing with water, sonicating and air drying, as performed previously.

### Results

The final functionalised cannula contained a thin layer of hyaluronic acid adsorbed to the fluoropolymer surface via a linker.

A protein adsorption test was performed to assess the impact of the hyaluronic acid species on the protein adsorption behaviour of the PTFE surface, in which the fluoropolymer surface of the cannula was treated with a bovine serum albumin (BSA) protein solution. BSA adsorption was assessed by fluorescence after 24- and 72-hours treatment.

The functionalised cannula demonstrated minimal fluorescence after both 24 and 72 hours of treatment, which suggested minimal protein adsorption had occurred on the functionalised surface. This was in stark contrast to an unmodified PTFE cannula control, which displayed substantial fluorescence after both time periods.

These results highlight the excellent protein-repellent properties provided by the hyaluronic acid functionalised PTFE surface. The mechanical properties of the PTFE cannula were not negatively impacted, and the PTFE retained its lubricious non-stick surface.

### EXAMPLE 2

A second embodiment of a medical device of the first aspect of the invention is provided by a cannula containing a polymeric tubular body having a PTFE outer surface. The PTFE outer surface is functionalised with hyaluronan, which is bonded to the PTFE surface via a linker derived from ethylenediamine.

The cannula is part of a patch pump of the second aspect of the invention for the subcutaneous delivery of insulin. A cross-sectional view of the patch pump is displayed in FIG. 3. With reference to FIG. 3, the patch pump comprises a body (101), which is attachable to the skin of a user via an adhesive part of the body (101). The patch pump comprises a cannula (105) which extends from the body (101).

The body (101) comprises a fluid part (107), which is part of the body and provides a fluid path through the patch pump, allowing for fluid communication between the body (101) and the cannula (105). The fluid part (107) also contains a cartridge of insulin (not shown) for subcutaneous delivery. The arrangement also allows for fluid communication between the inside of the insulin cartridge and the cannula (105).

The body (101) further comprises an inbuilt pump (not shown).

In use, the body (101) of the patch pump is attached to the skin of the user via the adhesive part of the body (101). The free end of the cannula (105), which projects from the body (101), is inserted into the body of the user. Insulin is delivered subcutaneously from the patch pump via the cannula (105), with assistance from the inbuilt pump.

### Cannula Preparation Method

The functionalised cannula was prepared as follows.

Surface Activation: surface activation was performed as described for Example 1 above.

Linker functionalisation: the cannula was thereafter submerged in neat ethylenediamine at room temperature for 1 hour. The cannula was then removed, rinsed with deionised water at ambient temperature, and sonicated for 10 minutes in fresh deionised water. The cannula was then air dried.

Hyaluronic acid functionalisation: a 1 wt. % solution of sodium hyaluronate (MWt 1.5-2.2 MDa) in deionised water was prepared. The cannula was submerged in the prepared solution at room temperature for 1 hour, prior to rinsing with water, sonicating and air drying, as performed previously.

### Results

The final functionalised cannula contained a thin layer of hyaluronic acid adsorbed to the fluoropolymer surface via a linker.

A protein adsorption test was performed as in Example 1, and results achieved were similar.

The mechanical properties of the PTFE cannula were not negatively impacted, and the PTFE retained its lubricious non-stick surface.

### EXAMPLE 3

A third embodiment of a medical device of the first aspect of the invention is provided by a cannula containing a polymeric tubular body having a PTFE outer surface. The PTFE outer surface is functionalised with hyaluronan, which is bonded to the PTFE surface via a linker derived from glycidyl methacrylate.

The cannula is part of an infusion set of the second aspect of the invention, as for Example 1 above.

### Cannula Preparation Method

The functionalised cannula was prepared as follows.

Surface Activation: the PTFE surface was treated with plasma comprising a helium primary gas at a flow rate of 15 Lpm and an oxygen secondary gas at a flow rate of 0.65 Lpm. Treatment was performed for 15-300 seconds at 60° C., and using radio waves having a power of 160 W.

Linker functionalisation: a 2 wt. % solution of glycidyl methacrylate in methyl tert-butyl ether was prepared. The cannula was submerged in the solution at room temperature for 1 hour. The cannula was then removed, rinsed with deionised water at ambient temperature, and sonicated for 10 minutes in fresh deionised water. The cannula was then air dried.

Hyaluronic acid functionalisation: the cannula is then treated with hyaluronic acid methacrylate (MWt 120000-150000; compound (I) below) in the presence of an azobisisobutyronitrile (AIBN) radical initiator to attach the hyaluronic acid species to the fluoropolymer surface via the attached linker.

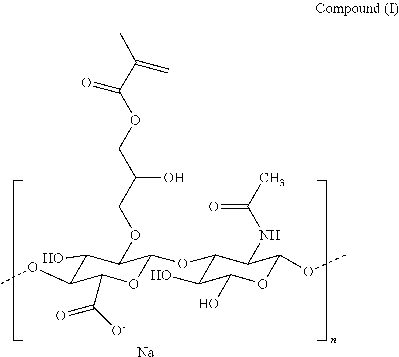

### Results

The final functionalised cannula contained a thin layer of hyaluronic acid species adsorbed to the fluoropolymer surface via a linker.

The functionalised cannula displayed minimal protein adsorption and the mechanical properties of the PTFE cannula were not negatively impacted.

The above embodiments are described by way of example only. Many variations are possible without departing from the scope of the invention as defined in the appended claims.

1. A medical device comprising a fluoropolymer surface comprising at least one hyaluronic acid species.

2. A medical device as claimed in claim 1, wherein the at least one hyaluronic acid species is present as a coating on the fluoropolymer surface.

3. A medical device as claimed in claim 1, wherein the fluoropolymer is independently chosen from: polytetrafluoroethylene, polyvinylfluoride, polyvinylidene fluoride, polychlorotrifluoroethylene, a perfluoroalkoxy polymer, fluorinated ethylene-propylene, polyethylenetetrafluoroethylene, polyethylenechlorotrifluoroethylene, a perfluoroelastomer, a fluoroelastomer, perfluoropolyether, perfluorosulfonic acid, perfluoropolyoxetane, and combinations, blends or copolymers thereof, and wherein the fluoropolymer comprises polytetrafluoroethylene.

4. A medical device as claimed in claim 1, wherein the at least one hyaluronic acid species comprises hyaluronan or a derivative thereof.

5. A medical device as claimed in claim 1, wherein the at least one hyaluronic acid species is adsorbed to the fluoropolymer surface, and wherein the at least one hyaluronic acid species is physisorbed and/or chemisorbed to the fluoropolymer surface.

6. A medical device as claimed in claim 1, wherein the at least one hyaluronic acid species is covalently bonded to the fluoropolymer surface.

7. A medical device as claimed in claim 1, wherein the at least one hyaluronic acid species is bonded to the fluoropolymer surface via at least one carboxyl group on the hyaluronic acid species.

8. A medical device as claimed in claim 1, wherein the at least one hyaluronic acid species is bonded to the fluoropolymer surface via a linker.

9. A medical device as claimed in claim 8, wherein the at least one hyaluronic acid species is bonded to the linker by a covalent or ionic/electrostatic bonding method and the linker is bonded to the fluoropolymer surface by either the same bonding method or an opposite bonding method.

10. (canceled)

11. A medical device as claimed in claim 8, wherein the linker is derived from a linking compound comprising either an amine group or a bi- or poly-functional molecule comprising at least two reactive functional groups.

12. (canceled)

13. A medical device as claimed in claim 8, wherein the linker is derived from a linking compound comprising an alkylenediamine.

14. A medical device as claimed claim 1, wherein the at least one hyaluronic acid species is bonded to the fluoropolymer surface via an ester and/or amide bond formed through at least one carboxyl group on the hyaluronic acid species and/or the at least one hyaluronic acid is bonded to the fluoropolymer surface via a linker and the hyaluronic acid species is bonded to the linker via an ester and/or amide bond formed through at least one carboxyl group on the hyaluronic acid species.

15. A medical device as claimed in claim 1, wherein the at least one hyaluronic acid species is present at a total concentration of at least 0.5 wt. % of the medical device.

16. A medical device as claimed claim 1, wherein the at least one hyaluronic acid species is present at and/or on at least 50% of the total area of the fluoropolymer surface.

17. A medical device as claimed in claim 1, wherein the medical device comprises a tubular body comprising the fluoropolymer surface.

18. A medical device as claimed in claim 17, wherein the fluoropolymer surface comprises an outer surface of the tubular body, and comprises at least 70% of the outer surface area of the tubular body.

19. A medical device as claimed in claim 1, wherein the medical device is a cannula that is part of an infusion set or patch pumper a catheter.

20. (canceled)

21. A medical device as claimed in claim 1, wherein the fluoropolymer surface is an activated fluoropolymer surface and the at least one hyaluronic acid species is covalently bonded to the activated fluoropolymer surface via at least one oxygen-containing moiety on the fluoropolymer surface and/or the at least one hyaluronic acid is bonded to the fluoropolymer surface via a linker and the linker is covalently bonded to the activated fluoropolymer surface via at least one oxygen-containing moiety on the fluoropolymer surface.

22. A medical device as claimed in claim 1, wherein the fluoropolymer surface is an activated fluoropolymer surface and the at least one hyaluronic acid species is bonded to the activated fluoropolymer surface via a hydrogen bonding interaction with at least one oxygen-containing moiety on the activated fluoropolymer surface and/or the at least one hyaluronic acid is bonded to the fluoropolymer surface via a linker and the linker is bonded to the activated fluoropolymer surface via a hydrogen bonding interaction with at least one oxygen-containing moiety on the activated fluoropolymer surface.

23. (canceled)

24. A method of manufacturing a medical device, the method comprising the steps of:
a. Providing a medical device comprising a fluoropolymer surface;
b. Activating the fluoropolymer surface; and
c. Functionalising the activated fluoropolymer surface with at least one hyaluronic acid species.

25-29: (canceled)
